(19) United States
(12) Patent Application Publication
(10) Pub. No.: US 2004/0106929 A1
(43) Pub. Date: Jun. 3, 2004

(54) Method and apparatus for performing an accurately sized and placed anterior capsulorhexis

(76) Inventor: Samuel Masket, Venice, CA (US)

Correspondence Address: Genesis LLP (attn: Rupak Nag), Suite 18, 725 Florida St., San Francisco, CA, 94110, US

(21) Appl. No.: 10/644,601

(22) Filed: Aug. 20, 2003

Related U.S. Application Data
(60) Provisional application No. 60/405,092, filed on Aug. 20, 2002.

(51) Int. Cl. A61F 9/00

(52) U.S. Cl. 606/107

(57) ABSTRACT

Methods and devices of performing an improved anterior capsulorhexis, wherein the capsulorhexis is more precisely sized, shaped and positioned on the eye lens capsule are described. A capsulorhexis, also referred to as a capsulotomy, is a procedure for creating an aperture in a lens capsule by making an incision. The incision should be of a certain size and shape and be positioned on the capsule as determined by the surgeon. An image projection device is used to project a visible target image on the anterior or posterior of the lens capsule as a guide or trackable image for the surgeon. Before a procedure begins, a target image is configured by a surgeon to have a desired size and shape. During the procedure, target image is projected onto a desired location on the surface of lens capsule. In another preferred embodiment, a template is configured to a desired shape and size by a surgeon to correspond to the appropriate capsulorhexis. Once configured, the template is inserted into the anterior chamber, using an appropriate delivery mechanism if necessary. The template is used to make a physical impression or mark on the lens capsule wherein the mark closely corresponds to the intended incision.

### CROSS-REFERENCE TO RELATED APPLICATIONS

This application claims priority under 35 U.S.C. §119 from provisional patent application filed Aug. 20, 2002, application Ser. No. 60/405,092, titled “Capsule Marking Technique for Performing Accurately Sized and Placed Anterior Capsulorhexis.”

### BACKGROUND OF THE INVENTION

1. Field of the Invention

The present invention relates generally to surgical methods and apparatus. More specifically, the invention relates to surgical devices and procedures performed on the eye involving either projecting an image on or marking a lens capsule for performing an accurately sized, shaped, and placed anterior capsulorhexis.

2. Discussion of Related Art

Anterior capsulorhexis, also referred to as anterior capsulotomy, is a surgical procedure performed on the eye wherein a portion of an eye lens capsule is removed thereby forming an aperture or capsulorhexis in the lens capsule. Continuous tear anterior capsulotomy is an integral procedure of modern cataract surgery and clear lens replacement surgery. Currently, mechanical surgical instruments, such as cystitomes and forceps and automated devices such as lasers, ultrasonic needles, and diathermy are used to perform anterior capsulorhexis in cataract surgery, clear lens replacement surgery and, in fewer cases, to create a posterior capsulorhexis. However, when performing these procedures, the size, shape, and positioning of the capsulorhexis are not determined by any of these surgical instruments or devices, but rather are estimated manually by the surgeon. Even in modern ocular surgery, manual estimation by the surgeon is acceptable and widely practiced when performing an anterior or posterior capsulorhexis.

However, based on investigations conducted by the inventor, the inventor believes that advancements in procedures for performing cataract surgery and clear lens replacement surgery will very likely require more precise measurements of the size, shape and positioning of the capsulorhexis than is presently expected from an experienced surgeon's manual estimation. For example, in one type of procedure, a new lens is placed in a lens capsule remnant. Current and future designs of these new intraocular lens will require that the positioning of the new lens in the capsule remnant be very precise, if not exact. Furthermore, certain late complications typical of refractive surgery may be reduced if the new lens implant is positioned on the lens capsule sac exactly as desired by the surgeon. Therefore, in order to optimize surgical outcomes in procedures involving the positioning of these newly designed intraocular lenses, it is beneficial to create an anterior or posterior capsulorhexis that is precisely sized, shaped, and positioned on the lens capsule.

### SUMMARY OF THE INVENTION

In one aspect of the present invention, a method for performing a capsulorhexis of a lens capsule using an image projection device having a tracking mechanism is described. A surgeon determines the best size, shape, and location of the capsulorhexis by examining numerous characteristics and factors known in the field of ocular surgery. The surgeon configures a target image to be projected on the lens capsule wherein the image corresponds to the size and shape of the capsulorhexis, i.e, the incision in the lens capsule. An image projection device containing a tracking mechanism and tracking sensors projects the target image, pre-configured by the surgeon, onto a selected location on the lens capsule. The target image is used as a guide for the surgeon in performing a capsulorhexis. The precise location of the capsulorhexis on the lens capsule is maintained during surgery and movement of the lens capsule by utilizing a tracking mechanism and one or more tracking sensors in the image projection device.

In another aspect of the present invention, lens capsule marking methods are described. In this aspect of the invention, some type of physical impression is made at a desired location on the lens capsule by a template pre-configured by the surgeon to correspond to the size and shape of the capsulorhexis. The template is inserted into the anterior chamber and is brought in physical contact with the lens capsule. Depending on the type of template, a delivery mechanism may or may not be needed for inserting the template into the anterior chamber. In one embodiment, the template is comprised of an appropriate autoclaveable material or any material that can be sterilized and is in the form of a circular or elliptical loop. In other embodiments the template may be disjointed, for example, creating two semi-circular or semi-elliptical loops. The loop is inserted into the anterior chamber via a hollow insertion tube. Regardless of the specific embodiment of the capsule marking aspect of the present invention, a physical impression is made on the lens capsule at a desired location by touching the capsule surface with a template configured to correspond to the intended incision on the lens capsule. In one specific embodiment, a dye is applied to a loop which is configured to the desired size and shape of the capsulorhexis by the surgeon. The loop is retracted into an insertion tube and extended from the tube once in the anterior chamber to mark the lens capsule. In yet another embodiment, the loop has shape memory thereby allowing the loop to reconfigure to the size and shape selected by the surgeon when extended from the insertion tube. Because the image target in this aspect of the invention is a physical mark on the lens capsule, the surgeon can track the mark, which stays at the same location on the lens capsule, during surgery when the lens capsule may move.

### BRIEF DESCRIPTION OF THE DRAWINGS

The invention will be better understood by reference to the following description taken in conjunction with the accompanying drawings in which:

FIG. 1 is a diagram of an image projection device for projecting a pre-configured target image onto a surface of a lens capsule and tracking the position of the image during movement of the lens capsule utilizing a tracking mechanism and tracking sensors in accordance with one embodiment of the invention.

FIG. 2 is a diagram of a lens capsule marking mechanism comprised of an insertion tube and a loop having shape memory used to create a visible mark corresponding to a selected size and shape of a capsulorhexis on the surface of a lens capsule in accordance with one embodiment of the present invention.

FIGS. 3A and 3B are diagrams of a plunger-type mechanism and a screw-type mechanism for retracting and extending the loop to and from the insertion tube.

### DETAILED DESCRIPTION

Reference will now be made in detail to preferred embodiments of the invention. Examples of the preferred embodiments are illustrated in the accompanying drawings. While the invention will be described in conjunction with these preferred embodiments, it will be understood that it is not intended to limit the invention to these embodiments. To the contrary, it is intended to cover alternatives, modifications, and equivalents as may be included within the spirit and scope of the invention as defined by the appended claims.

Devices for and methods of performing an improved anterior capsulorhexis, wherein the capsulorhexis is more precisely sized, shaped and positioned on the eye lens capsule are described in the various figures. A capsulorhexis, also referred to as a capsulotomy, is a procedure for creating an aperture in a lens capsule by making an incision. The incision should be of a certain size and shape and be positioned on the capsule as determined by the surgeon.

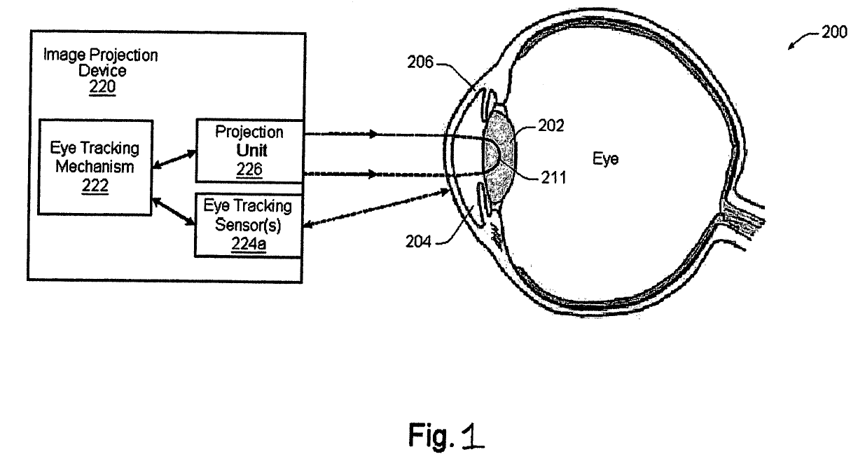

In a preferred embodiment of the present invention, an image projection device is used to project a visible target image on the anterior or posterior of the lens capsule as a guide or trackable image for the surgeon. Image projection devices are commonly used in lasic surgery, refractive or clear lens replacement surgery, or any type of surgery wherein an image is projected onto the cornea. Use of an image projection device to obtain a more accurately sized, shaped, and positioned anterior capsulorhexis is illustrated in FIG. 1. In FIG. 1 a conventional image projection device 220 projects a target image 211 onto the surface of the lens capsule 202. In this implementation, before a procedure begins, target image 211 is configured by a surgeon to have a desired size and shape. During the procedure, target image 211 is projected onto a desired location on the surface of lens capsule 202.

In another preferred embodiment of the present invention, a template is configured to a desired shape and size by a surgeon to correspond to the appropriate capsulorhexis. Once configured, the template is inserted into the anterior chamber via an appropriate delivery mechanism. The template is used to make a physical impression or mark on the lens capsule wherein the mark closely corresponds to the intended incision. In a specific embodiment, the template is composed of a deformable and malleable material such as stainless steel or titanium, and is in the form of a circular or elliptical loop. In one embodiment, the loop is retracted into a hollow insertion tube which is inserted into the anterior chamber. The loop is then extended from the tube to mark the lens capsule, whereby the mark is used as a guide for the surgeon for making the incision.

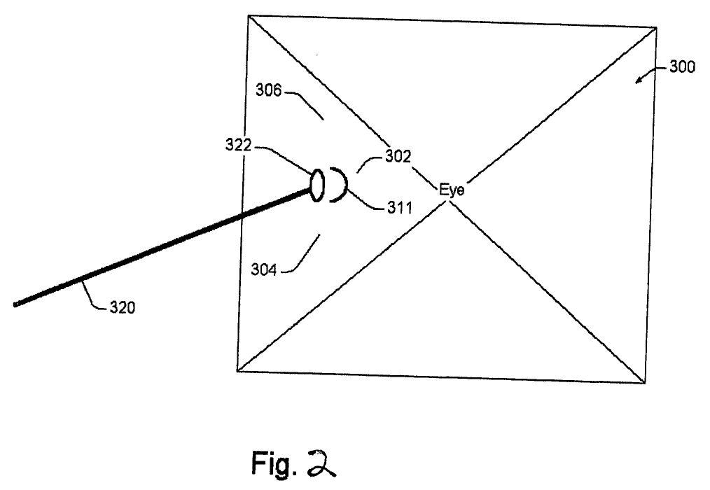

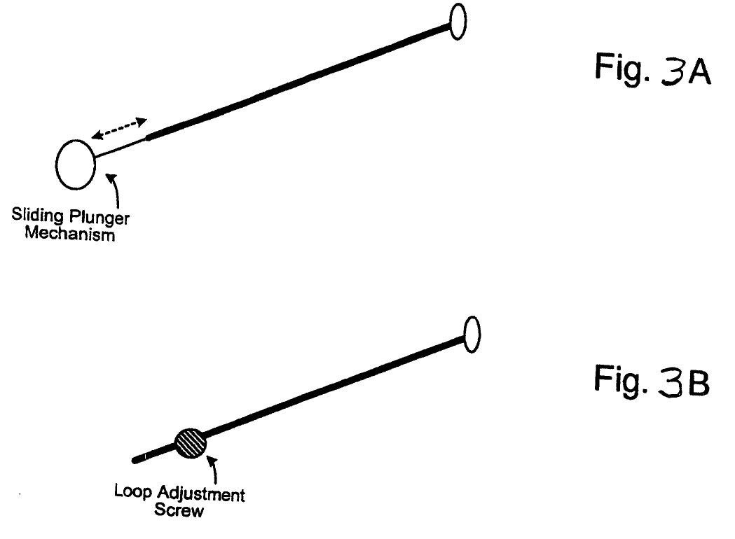

In one embodiment, the material used to form the loop is able to retain its shape and size once extended from the tube; that is, the material has what is referred to as shape memory. In one implementation, a dye is applied to the pre-configured loop before it is inserted into a tube. Once the tube is in the anterior chamber and the loop is extended from the tube, the loop physically marks or touches the lens capsule thereby marking the capsule at a selected position with an impression corresponding to the capsulorhexis. This implementation is shown in FIGS. 2, 3A and 3B.

In other embodiments the template, such as the loop, in some manner makes an impression on the capsule corresponding to the intended incision. In yet other embodiments, the template may be comprised of other types of materials for which a delivery mechanism such as an insertion tube may not be needed or for which certain properties, such as shape memory, may not be needed. The material should be autoclaveable so that it can be used in subsequent surgery. In this aspect of the invention, regardless of the specific implementation, the objective is to physically mark or create an impression on the lens capsule that corresponds to the intended capsulorhexis. By creating a physical impression on the capsule at the desired position, the surgeon has a target or point of reference for performing the capsulorhexis. Because the mark is a physical impression, the surgeon can keep track of the target position on the lens capsule without the need for any type of tracking mechanism or device.

Returning to the image projection method and device as shown in FIG. 1, image projection device 220 contains numerous components. These include at least one processor, memory, at least one interface for communicating with external electronic components, and a projection unit 226 such as a laser diode or other projection mechanisms commonly used in the field. Projected target image 211 is calibrated or configured to a desired size and shape by the surgeon. The surgeon may then adjust the position of projected target image 211 on lens capsule 202. In this embodiment, image projection device 220 is attached to an operating microscope (not shown).

As shown in FIG. 1 image projection device contains an eye tracking mechanism 222 coupled to eye tracking sensor(s) 224a which are utilized so that projected target image 211 remains at a fixed position relative to lens capsule 202 as the eye is surgically manipulated. Eye tracking mechanism and eye tracking sensors allow the surgeon to follow projected image 211 despite eye movement during surgical manipulation. In one implementation, a conventional type of eye tracking mechanism 222 may be used, such as those utilized in conventional laser eye surgery (e.g., the LADAR Vision tracking device distributed by Alcon Surgical of Fort Worth, Tex. and the VISX tracking device distributed by VISX of Santa Clara, Calif.).

In order to project a target image at a desired location on the lens capsule, various parameters may be considered and taken into account by the surgeon. These parameters include curvature of the eye, thickness of the cornea, depth of the anterior chamber, and other parameters. These parameters are also used by the surgeon to derive the optimal size and shape of the target image.

A preferred embodiment of a capsule marking method and device of the present invention are shown in FIGS. 2, 3A, and 3B. In this embodiment, an anterior capsule marking device 320 of FIG. 2 is used to mark the surface of a lens capsule 302, forming a pattern 311 having a selected size and shape which is used by the surgeon to perform the capsulorhexis procedure. In one implementation, capsule marking device 320 is comprised of a hollowed insertion tube into which a continuous loop 322 with shape memory may be retracted and extended. In another preferred embodiment, the loop is disjointed or split creating two semi-circular or semi-elliptical loops allowing for a narrower insertion tube. The loop is retractable into the insertion tube. The loop size is adjusted to pattern 311 corresponding to a desired size and shape of the anterior capsulorhexis.

In another preferred embodiment, loop 322 is extended during surgery, marked with a dye, and retracted into the insertion tube. Extension and retraction of loop 322 are accomplished via various mechanisms known in the field such as a plunger-type mechanism shown in FIG. 3A or a screw-type mechanism as shown in FIG. 3B. Once the loop is marked with an appropriate dye and retracted into the insertion tube, the tube is inserted into anterior chamber 304 of the eye through a small corneal incision, such as a standard cataract incision. Once in the anterior chamber, the loop is extracted from the tube and extended to its pre-configured size and shape, and used to make a mark on the anterior lens capsule at a desired position. The mark acts as a target or guide for the surgeon to follow in order to make a precise anterior capsulorhexis.

In one implementation, capsule marking device 320 is manufactured to create custom-sized loops of any desired size and shape as determined by the surgeon. For example, the shape of the loop may be substantially circular or elliptical.

In other preferred embodiments, the methods of the present invention may also be used in posterior capsulorhexis procedures. It may also be desirable to take into account optical properties of the ophthalmic visco surgical device (OVD) when projecting an image on the posterior of the lens capsule.

Generally, the image projection device embodiments of the present invention may be implemented by computer software, by computer hardware, or a combination of both. For example, the embodiments can be implemented in an operating system kernel, in a separate user process, in a library package bound into network applications, on a specially constructed machine, or on a network interface card.

The image projection device embodiment may be implemented in software such as an operating system or in a software application running above an operating system. A software or software/hardware hybrid implementation may be implemented on a general-purpose programmable computer selectively activated or reconfigured by a computer program stored in memory. Because data and program instructions may be used to implement the devices and methods described herein, the present invention may relate to machine-readable media that include program instructions, state information, and other data for performing various operations described herein.

Although several preferred embodiments of this invention have been described in detail herein with reference to the accompanying drawings, it is to be understood that the invention is not limited to these precise embodiments, and that various changes and modifications may be effected therein by one skilled in the art without departing from the scope or spirit of the invention.

What is claimed is:

1. A method for performing a capsulorhexis of a lens capsule, the method comprising:
configuring an image projection device to project a target image corresponding to a desired size and shape of a capsulorhexis;
projecting the target image to a specific location on the lens capsule; and
using the target image as a surgical guide to create a capsulorhexis of a desired size, shape, and location on the lens capsule.

2. A method as recited in claim 1 further comprising:
tracking the target image on the lens capsule using a tracking mechanism in the image projection device to address movement of the lens capsule during a procedure.

3. A method of performing a capsulotomy on a lens capsule comprising:
configuring a template to correspond to a specific shape and size of a capsulotomy as determined by a surgeon;
inserting the configured template into an anterior chamber;
creating a visual impression on a lens capsule at a specific location using the template whereby the impression corresponds to the specific size and shape of the intended capsulotomy.

4. A method as recited in claim 3 wherein inserting the configured template into the anterior further comprises:
utilizing a delivery mechanism to insert the configured template into the anterior chamber.
